(19) United States
(12) Patent Application Publication
(10) Pub. No.: US 2006/0276801 A1
(43) Pub. Date: Dec. 7, 2006

(54) Inter-cervical facet implant distraction tool

(76) Inventors: Scott A. Yerby, Montara, CA (US); Steven T. Mitchell, Pleasant Hill, CA (US); Charles J. Winslow, Walnut Creek, CA (US)

Correspondence Address: FLIESLER MEYER, LLP, FOUR EMBARCADERO CENTER, SUITE 400, SAN FRANCISCO, CA, 94111, US

(21) Appl. No.: 11/397,220

(22) Filed: Apr. 4, 2006

Related U.S. Application Data
(60) Provisional application No. 60/668,053, filed on Apr. 4, 2005.

(51) Int. Cl. A61B 17/58

(52) U.S. Cl. 606/90

(57) ABSTRACT

A distraction tool is disclosed which distracts, and preferably sizes, adjoining facets of a spine for an implant. The tool preferably includes a distraction head that has a first and a second head component. The first head component and the second head component preferably include inter-digitated fingers when the distraction head is in a non-distracted position. The tool includes a handle which is actuatable to move the distraction head to a distracted position, whereby the first set and second set of fingers are separated from one another. The tool can include a distraction gauge as well as a locking mechanism. The tool can also include a movement limitation mechanism to control the amount of distraction which the tool undergoes when actuated. The tool can include an insertion feature to allow the implant to be inserted into the facet joint while the tool is distracting the facets apart.

### PRIORITY CLAIM

This application claims priority to U.S. Provisional Patent Application No. 60/668,053, filed Apr. 4, 2005, entitled “INTER-CERVICAL FACET IMPLANT DISTRACTION TOOL” (KLYC-01095US2).

### RELATED APPLICATIONS

This patent application is related to the following applications, all of which are hereby incorporated herein by reference:

U.S. Application No. 60/635,453, entitled “INTER-CERVICAL FACET IMPLANT AND METHOD”, filed Dec. 13, 2004 [Atty. Docket No. KLYC-01118US0];

U.S. application Ser. No. 11/053,399, entitled “INTER-CERVICAL FACET IMPLANT AND METHOD”, filed Feb. 8, 2005 [Atty. Docket No. KLYC-01118US1];

U.S. application Ser. No. 11/053,624, entitled “INTER-CERVICAL FACET IMPLANT AND METHOD”, filed Feb. 8, 2005 [Atty. Docket No. KLYC-01118US2];

U.S. application Ser. No. 11/053,735, entitled INTER-CERVICAL FACET IMPLANT AND METHOD, filed Feb. 8, 2005 [Atty. Docket No. KLYC-01118US3]; and

U.S. application Ser. No. 11/053,346, entitled INTER-CERVICAL FACET IMPLANT AND METHOD, Feb. 8, 2005 [Atty. Docket No. KLYC-01122US0].

### FIELD OF THE INVENTION

The present invention relates to an inter-facet implant and a tool configured to allow insertion of the implant.

### BACKGROUND OF THE INVENTION

The spinal column is a bio-mechanical structure composed primarily of ligaments, muscles, vertebrae and intervertebral disks. The bio-mechanical functions of the spine include: (1) support of the body, which involves the transfer of the weight and the bending movements of the head, trunk and arms to the pelvis and legs, (2) complex physiological motion between these parts, and (3) protection of the spinal cord and the nerve roots.

As the present society ages, it is anticipated that there will be an increase in adverse spinal conditions which are characteristic of older people. By way of example only, with aging comes an increase in spinal stenosis (including, but not limited to, central canal and lateral stenosis), and facet arthropathy. Spinal stenosis results in a reduction foraminal area (i.e., the available space for the passage of nerves and blood vessels) which compresses the cervical nerve roots and causes radicular pain.

Another symptom of spinal stenosis is myelopathy, which results in neck pain and muscle weakness. Extension and ipsilateral rotation of the neck further reduces the foraminal area and contributes to pain, nerve root compression, and neural injury.

In particular, cervical radiculopathy secondary to disc herniation and cervical spondylotic foraminal stenosis typically affects patients in their fourth and fifth decade, and has an annual incidence rate of 83.2 per 100,000 people (based on 1994 information). Cervical radiculopathy is typically treated surgically with either an anterior cervical discectomy and fusion (“ACDF”) or posterior laminoforaminotomy with or without facetectomy. ACDF is the most commonly performed surgical procedure for cervical radiculopathy, as it has been shown to increase significantly the foramina dimensions when compared to the posterior laminoforaminotomy.

It is desirable to eliminate the need for major surgery for all individuals, and in particular, for the elderly. Accordingly, a need exists to develop spine implants and tools for successful insertion of the implants that alleviate pain caused by spinal stenosis and other such conditions caused by damage to, or degeneration of, the cervical spine. In particular, a need exists for a tool to distract the adjoining facets apart from each other to allow insertion of an inter-facet implant therebetween.

### BRIEF DESCRIPTION OF THE FIGURES

FIG. 1 illustrates a perspective view of the inter-facet implant in accordance with one embodiment of the present invention.

FIG. 2 illustrates a side view of the inter-facet implant inserted between two adjacent vertebral bodies in the cervical region of the spine in accordance with one embodiment of the present invention.

FIG. 3A illustrates a side view of a distraction tool in accordance with one embodiment of the present invention.

FIG. 3B illustrates a side view of the distraction tool in accordance with one embodiment of the present invention.

FIG. 4A illustrates a perspective view of a distraction head of the distraction tool in accordance with one embodiment of the present invention.

FIG. 4B illustrates a perspective view of the distraction head of the distraction tool in accordance with one embodiment of the present invention.

FIG. 5A illustrates a side view of a curved distraction head of the distraction tool in accordance with one embodiment of the present invention.

FIG. 5B illustrates a side view of the curved distraction head of the distraction tool in accordance with one embodiment of the present invention.

FIG. 6A illustrates a perspective view of a distraction tool in accordance with one embodiment of the present invention.

FIG. 6B illustrates a top view of the distraction tool in accordance with one embodiment of the present invention.

FIGS. 7A-7C illustrate one distraction process using the distraction tool of the present invention.

FIG. 7D illustrates a flow chart of one implantation method in accordance with one embodiment of the present invention.

FIG. 8A illustrates a perspective view of a distraction and insertion tool in accordance with one embodiment of the present invention.

FIG. 8B illustrates a top view of the distraction and insertion tool shown in FIG. 7A in accordance with one embodiment of the present invention.

FIG. 9 illustrates a perspective view of a distraction tool with sizing mechanism in accordance with one embodiment of the present invention.

### DETAILED DESCRIPTION OF THE PRESENT INVENTION

Embodiments of the present invention provide a tool for implanting a minimally invasive surgical apparatus that preserves the physiology of the spine. In particular, the tool preferably distracts the facets in the cervical spine to allow insertion of the implant, whereby the implant increases the foramina dimension in extension and neutral positions. Such implants distract, or increase the space between, the vertebrae to increase the foraminal area or dimension, and reduce pressure on the nerves and blood vessels of the cervical spine. In a specific preferred embodiment, an implanted inter-facet spacer of 1.5 mm to 2.5 mm in width can result in inter-facet distraction that increases foramina dimension in extension and neutral. Other inter-facet spacer dimensions also are contemplated by the invention described herein below.

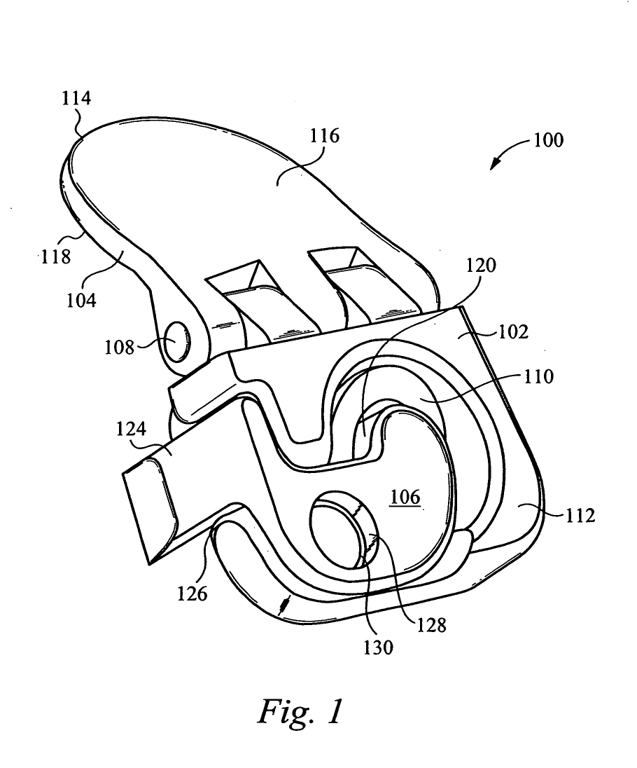

FIG. 1 illustrates a perspective view of an inter-facet cervical implant 100 in accordance with the present invention. In the embodiment depicted in FIG. 1, the implant 100 includes a lateral mass plate 102, an artificial facet joint 104 coupled to the mass plate 102 by a hinge 108, and a locking plate 106. As shown in FIG. 1, the mass plate 102 includes a recessed area 110 which receives the locking plate 106. The locking plate 106 is preferably of dimension such that the locking plate 106 is flush with the upper surface 112 of the lateral mass plate 102 when inserted therein. Other embodiments of the implant are discussed in U.S. Patent 60/635,453, which is incorporated by reference above.

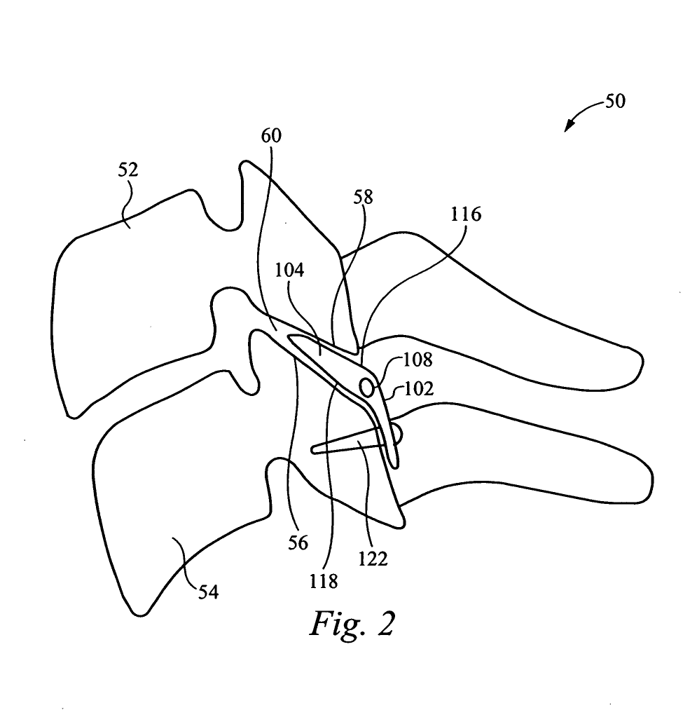

The artificial facet joint 104 in FIG. 1 is configured to fit between adjacent facets of the vertebral bodies, as shown in FIG. 2. In particular, the artificial facet joint 104 can fit the shape of a cervical facet joint 60, which is comprised of an inferior facet 58 of an upper vertebral body 52 and a superior facet 56 of a lower adjacent vertebral body 54. The superior surface 116 of the artificial facet joint 104 mates with the inferior facet 58 of the upper cervical vertebral body 52. The inferior surface 118 of the artificial facet joint 104 preferably mates with the superior facet 56 of the lower cervical vertebral body 54.

The shape of the artificial facet joint 104 can facilitate insertion of that portion of the implant 100 into the cervical facet joint 60. In the embodiment shown in FIG. 1, the artificial facet joint 104 has a rounded distal end 114, whereby the distal end 114 is preferably tapered in thickness to facilitate insertion. In one embodiment, the artificial facet joint 104 is curved downward, whereby its superior surface 116 is curved. The curve can cause the superior surface 116 to be convex, and the convexity can vary among different implants 1900 to suit the anatomical structure of the cervical facet joint(s) of a patient. An inferior surface 118 can accordingly be concave, flat, or convex in shape.

As stated above, the artificial facet joint 104 is connected with the lateral mass plate 102 by a hinge 108, whereby the hinge 108 allows the lateral mass plate 102 to bend at a wide range of angles relative to the artificial facet joint 104, preferably at an angle of more than 90 degrees. This flexibility facilitates positioning and insertion of the artificial facet joint 104 since the facet joints 60 can be highly variable among individuals. The hinge 108 enables positioning of the artificial facet joint 104 to be inserted into the facet joint 60 while the lateral mass plate 102 is moveable to conform to the patient's cervical spinal anatomy. In particular, the lateral mass plate 102 is positioned outside of the facet joint 60 and preferably against the lateral mass or lamina of the vertebral body when the artificial facet joint 104 is inserted between the facets. The lateral mass plate 102 has a bore 120 which passes therethrough. The bore 120 preferably accepts a bone screw 122 (FIG. 2), also referred to as a lateral mass screw, to secure the lateral mass plate 102 to the spine and thus to anchor the implant 100.

The implant 100 preferably includes a locking plate 106 which couples to the lateral mass plate 102, as shown in FIG. 1. The locking plate 106 preferably includes a keel 124 with a wedge shaped distal end to anchor the implant 100 preferably into the lateral mass or the lamina portion of the spine. The keel 124 preferably prevents rotation of the lateral mass plate 102 as well as the locking plate 106 when implanted. The keel 124 aligns with a groove 126 at a side of the lateral mass plate 102 to guide and align the keel 124 as the keel 124 is cut into the bone. The locking plate 106 preferably includes a probe 120 that fits into a bore in the lateral mass plate 102, as shown in FIG. 1. The locking plate 106 preferably also includes a bore 128 that can accept a machine screw (not shown) which passes through to an aligned bore 130 in the lateral mass plate 102 to hold the locking plate 106 and the lateral mass plate 102 together.

FIG. 2 illustrates the implant 100 inserted within the facet joint 60 between the adjacent vertebral bodies 52 and 54. As shown in FIG. 2, the artificial facet joint 104 includes the superior facet surface 116 as well as the inferior facet surface 118, whereby the superior surface 116 of the artificial facet joint 104 preferably mates with the inferior facet 58 of the upper vertebral body 52. Additionally, the inferior surface 118 of the artificial facet joint 104 preferably mates with the superior facet 56 of the lower vertebral body 54. As shown in FIG. 2, the lateral mass plate 102 is shown anchored to the lateral mass with a screw 122.

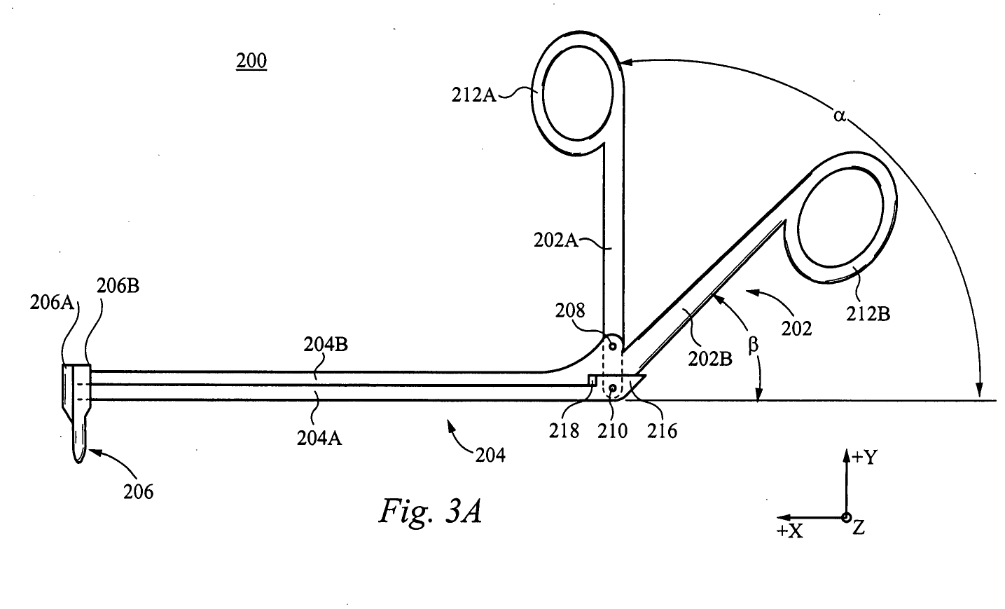

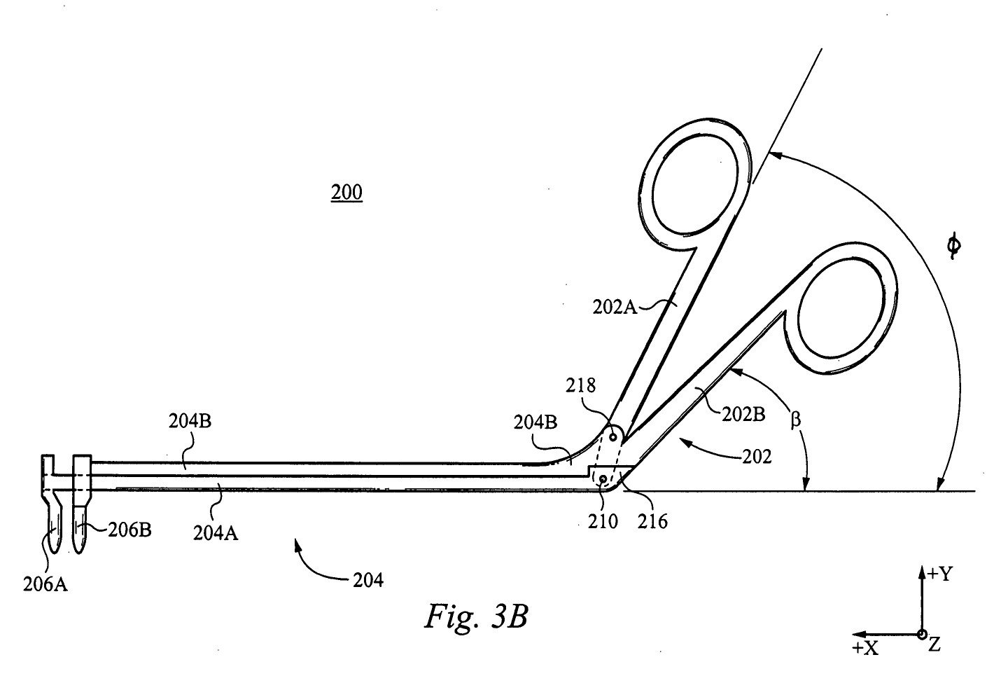

FIG. 3A illustrates a side view of a distractor tool in accordance with one embodiment of the present invention. As shown in FIG. 3A, the distractor tool 200 preferably includes a handle portion 202, an arm portion 204, and a distractor head portion 206. In particular, the handle portion 202 preferably includes a first handle 202A and a second handle 202B. The proximal ends of each handle 202A, 202B preferably include finger loops 212A and 212B, respectively. The handles 202A and 202B are coupled to one another at a pin 208. In a preferred embodiment, the first handle 202A is moveable whereas the second handle 202B is stationary with respect to the first handle 202A. In another embodiment, the second handle 202B is able to be pivotably rotated with respect to first handle 202A about pin 208. Alternatively, both handles are movable with respect to one another about pin 208.

As shown in the embodiment in FIG. 3A, the arm portion 204 has a first arm 204A and a second arm 204B. The arms 204 are oriented longitudinally along the X-axis. The upper arm 204B is preferably attached to the second handle 202B. However, the second arm 204B can alternatively be attached to the first handle 202A. In the embodiment in FIG. 3A, the first arm 204A and the second handle 202B are of one formed piece. Alternatively, the first arm 204A and the second handle 202B are two separate pieces which are coupled together.

As stated above, the first handle 202A is rotatable about pin 208, whereby the pin 208 is preferably located between the midpoint and a distal end of the handle 202A. In one embodiment shown in FIG. 3A and 3B, a proximal end of the first arm 204A is coupled to the distal end of the first handle 202A at pin 210. In another embodiment, the distal end of the handle 202A is coupled to an intermediate link which couples the handle 202A to the first arm 204A.

The first handle 202A is preferably moveable about pin 208 between an non-distracted position, as shown in FIG. 3A, and a distracted position, as shown in FIG. 3B. As shown in FIG. 3A, the first handle 202A is oriented at angle α with respect to the X-axis. In addition, the second handle 202B is oriented at angle β with respect to the X-axis. In FIG. 3A, the angle α of the first handle 202A in the non-distracted position is greater than the angle φ of the first handle 202A in the distracted position. It is preferred that, as the handles 202A, 202B are squeezed together, the tool 200 actuates from an non-distracted position to a distracted position.

When the handles 202A, 202B of the tool 200 are squeezed together, the clockwise rotational movement of the handle 202A about the pin 208 causes the distal end of the handle 202A to move the first arm 204A longitudinally along the positive X-axis (FIG. 3B). In contrast, when the handle 202 is released or when manually actuated to the non-distracted position, the counter-clockwise rotational movement of the handle 202A causes the distal end of the handle 202A to move the first arm 204A in the opposite direction, along the negative X-axis (FIG. 3A). The longitudinal movement of the first arm 204A along the X-axis causes the distraction head 206 to actuate and thus separate adjacent facets apart to allow implantation of the implant 100.

The distal ends of the first and second arms 204A, 204B are coupled to the distraction head 206 as shown in FIGS. 3A and 3B. The distraction head 206 preferably includes a first distraction head component 206A and a second distraction head component 206B. In one embodiment, the distal end of the first arm 204A is coupled to the first distraction head component 206A and the first distal end of the second arm 204B is coupled to the second distraction head component 206B. In another embodiment, the distal end of the first arm 204A is coupled to the second distraction head 206B and the distal end of the second arm 204B is coupled to the first distraction head 206B. Since the first arm 204A is attached to the first distraction head component 206A, the movement of the first arm 204A along the X-axis preferably causes the first distraction head component 206A to also move along the X-axis. The second head component 206B is preferably fixed to the second arm 204B. Therefore, the movement of the arm 204 along the positive X-axis causes the first head component 206A to move preferably away from the second head component 206B. The first head component 206A and the second head component 206B preferably separate the adjacent facets apart between 1.5 and 2.5 mm to accommodate the thickness of the artificial joint facet 104 of the implant 100. However, other distances are contemplated and are not limited to that described above.

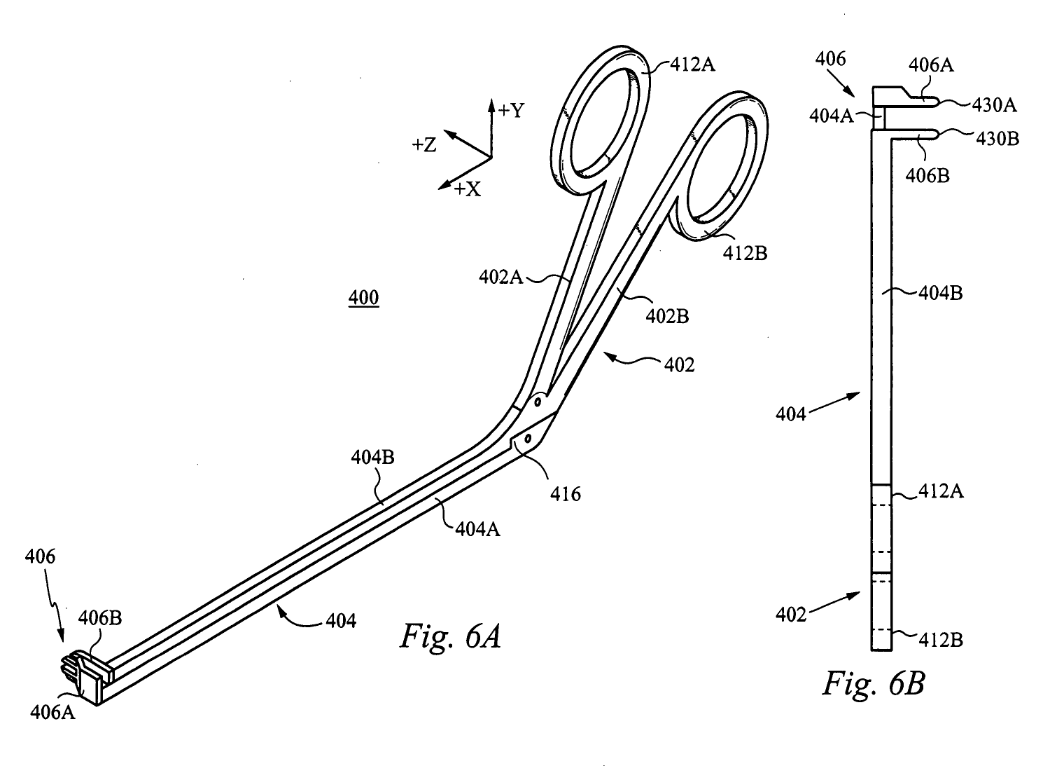

In the preferred embodiment, the distal portion of the distraction head extends substantially perpendicular to the arms 204A, 204B, as shown in FIGS. 3A and 3B. In an embodiment of the invention, head components 206A, 206B remain parallel with respect to each other in the open position as shown in FIGS. 3A and 3B. In another embodiment, the superior and inferior surfaces of the distraction head extend at an angle other than 90 degrees from the arms 204A and 204B. In the preferred embodiment shown in FIGS. 3A and 3B, the head components 206A, 206B of the distraction head 206 are oriented such that the leading edge 230 extends in the negative Y direction. Alternatively, the distraction head 206 is oriented such that the leading edge faces the positive Y direction. However, it is contemplated that the distraction head 206 can be oriented to extend from the arm 202 such that the leading edge faces the Z direction, as shown in FIGS. 6A and 6B. It is contemplated that the leading edge 230 of the distraction head 206 of the present invention can face any direction with respect to the arms 204 and the handles 202 including the negative Z direction.

The tool 200 of the present invention is preferably made from a medical grade metal. For example, the tool 200 can be made of titanium, stainless steel, an alloy or any other material which provides the tool 200 with a sufficient amount of strength to distract the adjacent facets apart during the implantation process. In one embodiment, the distraction head 206 is removable from the distal ends of arms, such that different sized distraction heads can be used with the same tool. This feature would allow the surgeon to replace the distraction head with one of a different size for a different inter-cervical facet joint without having to use a different tool. In another embodiment, the distraction head 206 is mounted to the arms 204 of the tool 100, whereby the upper head component 206A is welded to the lower arm 204A and the lower head component 206B is welded to the upper arm 204B or vice versa. Any other appropriate method of attaching the distraction head 206 to the arms 204 is contemplated.

It is preferred that the tool 200 includes a movement limitation mechanism. The mechanism preferably limits the amount of distraction between the first and second head components 206A, 206B when the handles 202 are actuated. As shown in FIGS. 3A and 3B, the proximal end of the first arm 204A preferably has a wedge-shaped portion 216. In addition, the second arm 204B includes a correspondingly shaped slot 218 which receives the wedged portion 216 during movement of the wedged portion 216 in the positive X direction. The slot 218 limits longitudinal movement of the first arm 204A along the X-axis when the handles 202 are squeezed. This, in effect, limits the distance that the head components 206A, 206B separate in distracting the facets apart from one another during the implantation procedure. Alternatively, any other mechanism is contemplated to limit movement of the distraction head 206 and is not limited to the wedged portion 216 and corresponding slot 218 of the present tool. It should be noted that the movement limitation mechanism is alternatively not incorporated in the tool of the present invention.

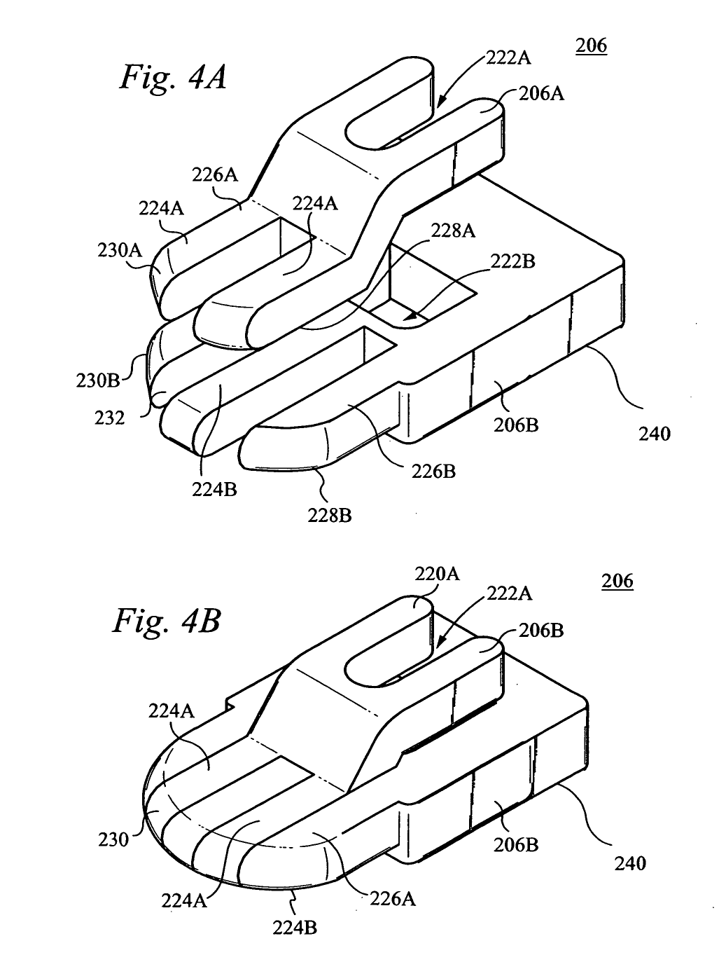

FIG. 4A illustrates a perspective view of the distraction head 206 in a distracted position in accordance with one embodiment. FIG. 4B illustrates a perspective view of the distraction head 206 in FIG. 4A in a non-distracted position. As shown in FIGS. 4A and 4B, the distraction head 206 preferably includes the first head component 206A having a proximal portion and a distal portion as well as the second head component 206B having a proximal portion and a distal portion. As shown in FIGS. 4A and 4B, the first head component 206A includes an engagement slot 222A at the proximal end. In addition, the second head component 206B includes a pass-through slot 222B which is aligned with the engagement slot 222A. The engagement slot 222A of the first head component 206A preferably receives and mounts to the distal end of the first arm 204A. The first arm 204A preferably extends through the pass-through slot 222B in the second head component 206B to allow the arm 204A to freely move the first head component 206A without interfering with the second head component 206B. The proximal portion of the second distraction head 206B is attached to the distal end of the second arm 204B. The second arm 204B is preferably mounted to the underside 240 of the second head component 206B, whereby the second arm 204B is located adjacent to the first arm 204A. It should be noted that the above description of the head components is preferred and can have any other appropriate configuration to allow distraction in accordance with the present invention.

The distal portion of both first and second distraction heads 206A, 206B includes leading edges, shown as 230A and 230B, which are used to penetrate the facet joint to insert the distraction head 206 therein. The distal portion of the first and second head components, as shown in FIG. 4A, include several fingers which are shown alternately arranged. In particular, the first distraction head 206A is shown to have two fingers 224A whereas the second distraction head 206B is shown to have three fingers 224B. In another embodiment, the upper and lower distraction heads 206A, 206B have a greater or fewer number of fingers than that shown in FIG. 4A, including only one finger each. The fingers 224A, 224B together form an overall rounded leading edge 230 of the distraction head 206 as shown in FIG. 4B. In another embodiment, the leading edges 230 of the fingers do not form a rounded leading edge, but can form any other shape.

As shown in FIGS. 4A and 4B, the second head component 206B includes finger slots 232 which receive the fingers 224A of the first head component 206A when the distraction head 206 is in the non-distracted position (FIG. 4B). In the non-distracted position, as shown in FIG. 4B, the first head component 206A and the second head component 206B are co-planar, whereby the fingers 224A and 224B are preferably inter-digitated. The co-planar head components provide a height dimension or thickness which allows the distraction head 206 to be easily inserted into the facet joint. Upon the handles 202 being squeezed, the first head component 206A is forced away from the second head component 206B, thereby causing the first set of fingers 224A from sliding out of the finger slots 232 of the second head component 206B. The first head component 206A thus moves apart from the second head component 206B until the desired distance between the head components is achieved. As shown in FIG. 4A, the fingers 224A of the first head component 206A are separated from the fingers 224B of the second head component 206B and is no longer co-planar in the distracted position.

As shown in FIG. 4A, the fingers 224A, 224B each have a superior surface 226A, 226B, as well as an inferior surface 228A, 228B. In one embodiment, the leading edge 230A, 230B of the fingers 224A, 224B are rounded or curved, as shown in FIGS. 4A and 4B. In another embodiment, the leading edges of the fingers 224A, 224B are sharpened.

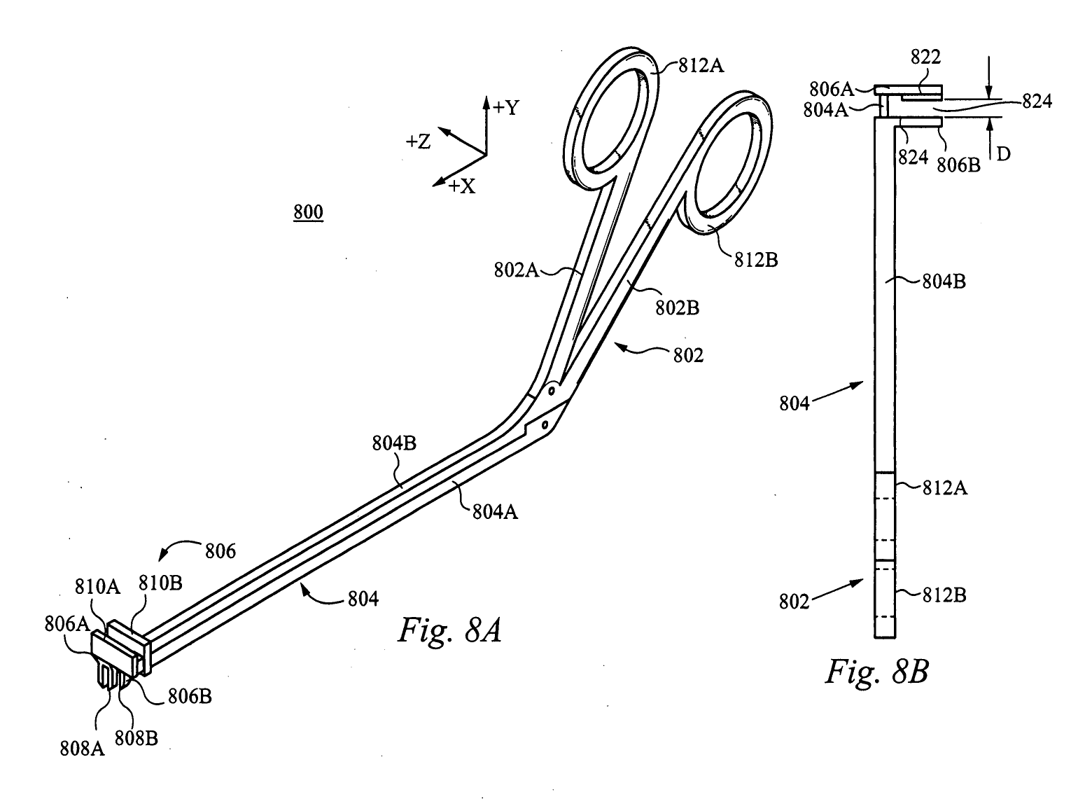

In one embodiment, the superior surfaces 226A, 226B of the distraction head components 206A, 206B mate with the inferior facet 58 of the vertebral body 52 when the distraction head 206 is inserted into the facet joint (FIG. 2). Additionally, in one embodiment, the inferior surfaces 228A, 228B of the distraction heads 206A, 206B mate with the superior facet 56 of the vertebral body 54. However, it is contemplated that the tool 200 can be oriented upside down such that the superior surface of the head 206 mates with the superior facet and the inferior surface of the head 206 mates with the inferior facet of the vertebral bodies 52, 54, as shown in FIGS. 8A-8C.

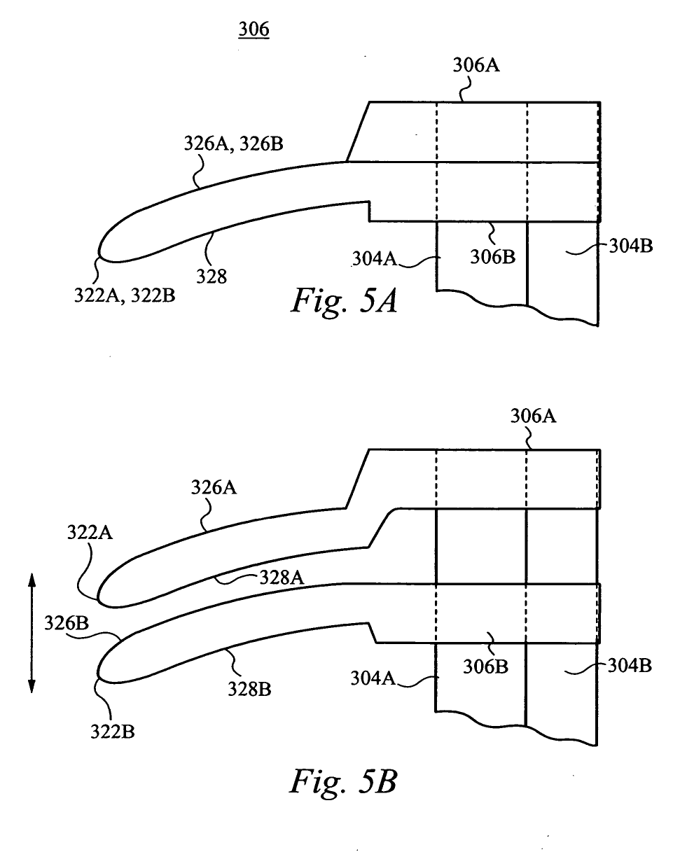

As shown in FIGS. 4A and 4B, the distal portion of the distraction head 206 is relatively flat such that the superior and inferior surfaces 226, 228 of the head components 206A, 206B are generally parallel with one another and have a uniform thickness. In another embodiment, the inferior and superior surfaces taper toward each other at the leading edge 230A, 230B. The head components 306A, 306B can alternatively be shaped to contour the shapes of the facets. The facet itself is somewhat shaped like a ball and socket joint. Accordingly, as depicted in FIGS. 5A and 5B, the distraction head 306 can have a convex superior surface 326 and a concave inferior surface 328. The curved superior and inferior surfaces preferably taper toward each other at the leading edge 322A, 322B to facilitate insertion, while the remainder of the distraction head has a uniform thickness.

In addition, as shown in FIG. 5B, the individual head components (FIG. 5B) each can have a concave and/or convex shape. In another embodiment, one of the superior and inferior surfaces 326A, 326B, 328A, 328B have a convex or concave shape, whereas the other surface is planar and does not have a curved shape. The superior and inferior surfaces of the distraction head 306 thus preferably contour the respective facets of the joint. The contour of the superior and/or inferior surfaces of the head 306 allows the upper and lower head components to apply a relatively constant force to the superior and inferior facets while the tool is actuated to the distracted position. In addition, the contoured shaped of the distraction head 306 along with its fingers allow the head components to obtain a better grip with their respective facets during the distraction procedure.

FIGS. 6A and 6B illustrate another embodiment of the tool having the distraction head in an alternative orientation than that shown in FIGS. 3A and 3B. As shown in FIG. 6A, the tool 400 includes the handle portion 402, the arm section 404 and the distraction head 406. As shown in FIG. 6A, the arm portion 404 is oriented along the X-axis. However, unlike the tool 200 described in FIGS. 3A and 3B, the distraction head 406 extends from the arm portion 404 such that the leading edge 430 faces in the positive Z direction. In the embodiment shown in FIG. 6, the distraction head 406 extends from the arm portion along the positive Z direction at approximately a 90 degree angle with respect to the arm 404. However, the distraction head 406 can be oriented to extend from the arm 404 along the negative Z direction or at any other angle besides 90 degrees.

In operation, actuation of the handle 402A causes the arm 404A to move along the X axis to actuate the distraction head 406 as shown in FIG. 6B. As shown in FIG. 6B, the leading edges 430A and 430B of the first and second head components 406A, 406B are preferably tapered. The orientation of the leading edge 230 in the Z direction allows the tool 400 to be oriented in a different manner than the tool 200 in FIGS. 3A and 3B during the implantation procedure. This alternative orientation of the tool 400 may be advantageous to distract facets along different portions of the spine which require the tool 400 to be oriented at a different angle. Additionally, the individual tastes of each physician may prefer the alternative orientation of the tool 400 over the orientation of the head 206 in the embodiment in FIGS. 3A and 3B.

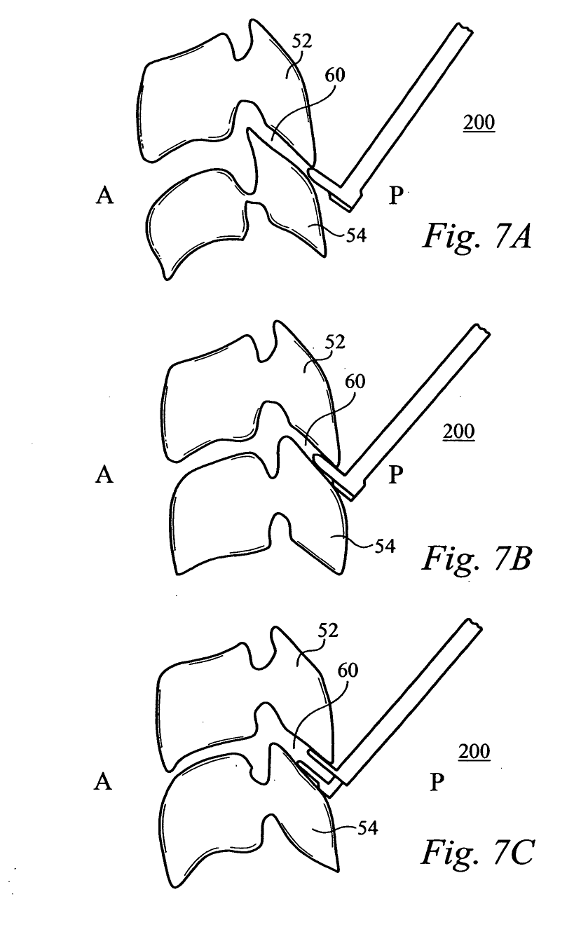

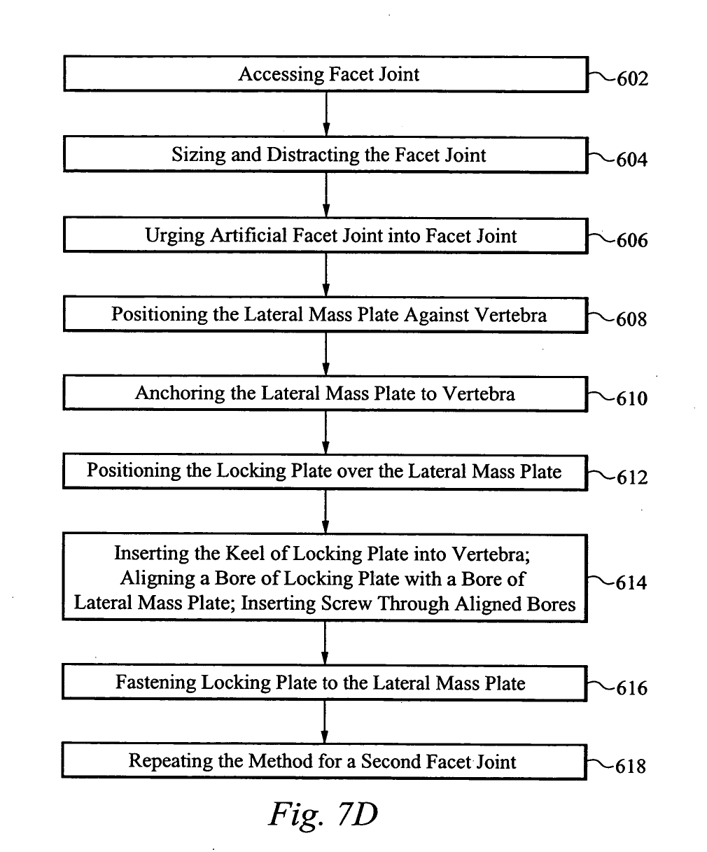

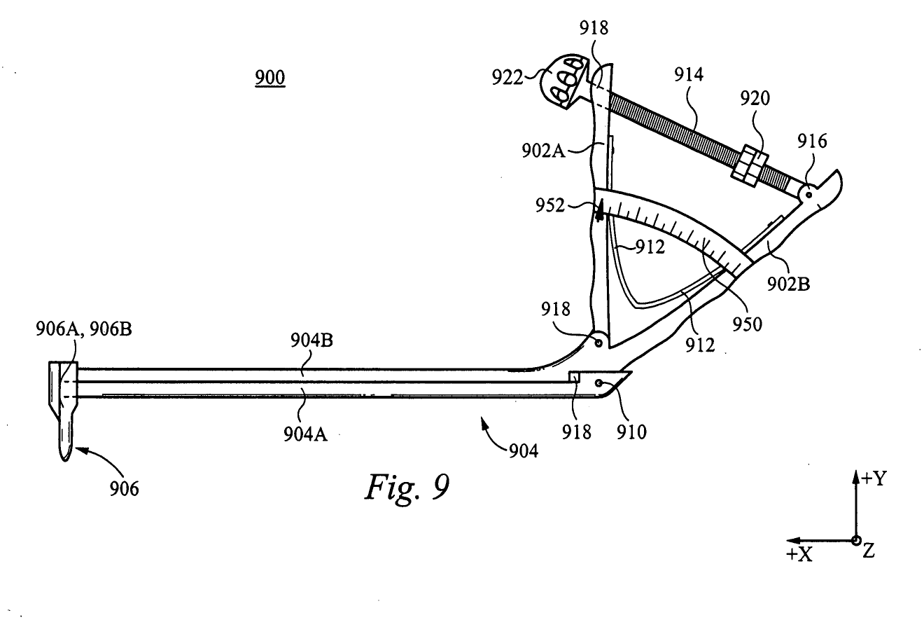

FIGS. 7A-7C illustrate one method of distracting adjacent facets in accordance with the tool of the present invention. FIG. 7D illustrates a flow chart of the method of implantation in accordance with one embodiment of the invention. The facet joint 60 is initially accessed as in step 602, as shown in FIG. 7A. A sizing tool can be inserted into the facet joint 60 to select the appropriate size of implant to be inserted as in step 604. In one embodiment, the sizing tool is a unit separate from the tool 200 of the present invention. In another embodiment, the tool 200 of the present invention has a sizing gauge to allow the surgeon to determine what size of implant 100 is to be inserted into the facet joint as discussed in relation to FIG. 9. As shown in FIG. 7A, the leading edge 230 of the tool 200 is then inserted into the entrance of the facet joint 60. The leading edge 230 of the tool 200 is then urged into the facet joint 60 until the distraction head 206 is sufficiently displaced within the facet joint 60 and between the superior and inferior facets 56, 58, as in FIG. 7B. In FIGS. 7A-7C, the tool 200 accesses the joint from a superior approach (i.e. upside down). However, it should be noted that the tool 200 can alternatively access the facet joint from an inferior (e.g. right side up) or lateral (e.g. sideways) approach.

Once the distraction head 206 is inserted, the physician squeezes the handles 202A, 202B together, whereby the distraction head components 206A and 206B separate from one another and distract the facet joint and surrounding tissue in order to facilitate insertion of the implant, as in step 604 (FIG. 7C). Once the adjacent facets are distracted apart the desired distance, the tool 200 is then removed from the joint, thereby leaving the adjacent facets apart from one another. The distracted tissue surrounding the facets slowly contract, thereby leaving time for the physician to urge the artificial facet joint 104 of the implant 100 between the facets into the facet joint, as in step 606.

Once the artificial joint 104 is inserted, the lateral mass plate 102 of the implant 100 is pivoted downward about the hinge 108 toward the lateral mass or to the lamina, as in step 608. Once the lateral mass plate 102 is positioned, or prior to the positioning of the lateral mass plate 102, a bore can be drilled into the bone to accommodate the bone screw 122. The screw is then placed through the bore 120 and secured to the bone to anchor the artificial facet joint 104 in place as in step 610. In order to lock the bone screw 122 and position of the artificial facet joint 104 and lateral mass plate 102 in place, the locking plate 106 is positioned over the lateral mass plate 102, as in step 612. The keel 124 located adjacent to the locking plate 106 can preferably self-cut a groove into the bone to lock the keel 1828 and anchor the implant 100, as in step 614. The locking plate 106 is then fastened to the lateral mass plate with the screw through the bore 130, as in step 616. This method is then repeated for any other facet joints in the spine, as in step 618.

FIGS. 8A and 8B illustrate another embodiment of the tool of the present invention. The embodiment shown in FIGS. 8A and 8B includes a distraction head 806 which is configured to distract adjacent facets of the vertebral bodies and simultaneously allow insertion of the implant (FIG. 1) into the facet joint 60. The tool 800 shown in FIGS. 8A and 8B includes the handle portion 802, the arm portion 804 as well as the distraction head 806.

As shown in FIGS. 8A and 8B, the fingers of the distraction head 806 are offset and adjacent to the arms 804A and 804B of the tool 800. As shown in FIGS. 8A and 8B, the distraction head 806 includes a leading edge 808 which is shown facing the negative Y direction as well as an insertion edge 810 which faces the positive Y direction. The insertion edge 810 is preferably located on the opposite end of the head 806 from the leading edge 808. The leading edge 808 is configured to be inserted into the facet joint 60 to distract the adjacent facets apart as stated above. The insertion end 810, upon distraction, allows the implant 100 (FIG. 1) to be inserted into the facet joint 60 while the tool 200 is simultaneously distracting the facets apart. The insertion edges 810A, 810B of the head components 806A, 806B, respectively, move apart as the head components 806A, 806B are distracted. This creates an insertion conduit 824 between (FIG. 8B) the first and second head components 806A, 806B. The insertion conduit 812 has a height distance, D, which provides adequate clearance between the inferior surface 822 of the first head component 806A and the superior surface 824 of the second head component 804B to allow the implant 100 to be inserted therethrough. As stated above, the distraction head 806 is offset and located adjacent to the arms 804 and handle 802 of the tool 800, whereby the location of the head 806 provide ample room to insert the implant 100 therethrough.

In operation, upon the distraction head 806 being inserted into the facet joint 60, the handles 802 are squeezed together to cause the distraction head components 806 to separate, thereby distracting the facets until the insertion conduit 812 is at the desired height dimension D. The desired height dimension, D, will depend on several factors, such as size of the artificial inter-facet joint 104, the thickness of the fingers of the head components, and the location of the facet joint (e.g. cervical, thoracic, lumbar). It is preferred that the height dimension D be between 1.5 and 2.5 mm, although other dimensions are contemplated. The height dimension D can be measured by a distraction gauge, as stated below, to achieve the desired height dimension.

Upon achieving the desired height dimension, D, the artificial insertion joint 104 of the implant 100 is inserted into the insertion conduit 812 via the insertion end 810. Considering that the insertion conduit 812 is in communication with the facet joint 60 of the spine, the implant 100 is able to slide through the conduit 812 into the facet joint 60. Upon the artificial inter-facet joint 104 being secured in the facet joint 60, the distraction head 806 can then be removed from the facet joint 60, thereby leaving the implant 100 inserted therein. The implant 100 can then be anchored as discussed above.

This embodiment allows the physician to maintain the distraction distance between the facets while inserting the implant 100. This embodiment, including the sizing gauge discussed below, can allow the physician to size, distract, and insert the implant using one tool. It should be noted that although the embodiment in FIG. 7A has the lead and insertion edges of the distraction head facing in the Y direction, the lead and insertion edges can face the Z direction or any other direction.

In one embodiment shown in FIG. 9, the distraction tool 900 can include a sizing mechanism in accordance with one embodiment of the present invention. As shown in FIG. 9, the distraction gauge 950 is coupled to one of the handles 902A and 902B. The other handle can include a flag 952 or pointer for indicating a distraction height measurement on the distraction gauge 950. Thus, as the handle 902A is urged toward the distraction position, the distraction gauge 950 slides past the flag 952, along with indicia indicating the increasing distraction height, D, between the distraction head components 906A and 906B.

In one embodiment, the distraction gauge 950 is configured to provide the amount of distance between the inferior surface of the first head component 906A and the superior surface of the second head component 906B (i.e. the insertion conduit). In another embodiment, the distraction gauge 950 can be configured to include the thickness of the first and second head components and thereby indicate the total distraction distance between adjacent facets.

In one embodiment, the tool 900 includes a spring mechanism to urge the handles 902A, 902B apart toward the non-distracted position. For example, a leaf spring 912 can be configured along the inner surfaces of the handles 902A, 902B to provide an outward bias against the handles 902A, 902B. In another example, a spring can be positioned between the interior wall of the slot 918 and the wedge portion 916 of the arm 904A to urge the wedged portion 916 and thus the handle 902A toward the non-distracted position.

Additionally, or alternatively, the tool 900 can include a locking mechanism to lock the tool 900 in a desired position. For example, the locking mechanism can include a threaded rod 914 which is coupled to one of the handles 902A, 902B at a pivot point 916, whereby the rod 914 freely passes through a through-hole in the other of the first and second handles 902A, 902B. The rod 914 includes a turning bolt 922 on the outer surface of the handle 904A which limits movement of the handles 902 which is caused by the force of the spring 910. As the handle 902A is urged closed, the threaded rod 914 passes through the through-hole and pivots to follow the arcing travel of the handle 902A. A distraction stop 920 can be positioned along the threaded rod 914 and sized such that the distraction stop 920 blocks the free travel of the threaded rod 914, thereby preventing further movement of the handle 902 and limiting the distraction height. In one embodiment, the distraction stop 920 is fixed in position along the threaded rod 914, however, in other embodiments the distraction stop 920 can be adjustably positionable along the threaded rod 914 to allow the maximum distraction height to be adjusted.

The foregoing description of preferred embodiments of the present invention has been provided for the purposes of illustration and description. It is not intended to be exhaustive or to limit the invention to the precise forms disclosed. Many modifications and variations will be apparent to one of ordinary skill in the relevant arts. The embodiments were chosen and described in order to best explain the principles of the invention and its practical application, thereby enabling others skilled in the art to understand the invention for various embodiments and with various modifications that are suited to the particular use contemplated. It is intended that the scope of the invention be defined by the claims and their equivalence.

What is claimed is:

1. A distraction tool to distract adjacent facets in a spine for insertion of an implant comprising:
a. a distraction head having a first head component and a second head component;
b. Two or more fingers extending from the first head component and one or more fingers extending from the second head component; wherein the two or more fingers of the first head component inter align with the one or more fingers of the second head component allowing the first head component and the second head component to be coplanar in the closed position; and
c. an actuatable handle coupled to the distraction head, wherein the plurality of fingers of the first head component and the plurality of fingers of the second head component are non-coplanar when the handle is operated to actuate the first and second head components to an open position.

2. The tool of claim 1, wherein the handle includes a first arm coupled to the first head component and a second arm coupled to the second head component, wherein the first arm longitudinally moves in relation to the second arm when the handle is actuated.

3. The tool of claim 1, wherein the distraction head is circular.

4. The tool of claim 1, wherein the plurality of first head component fingers and the plurality second head component fingers remain approximately parallel to each other in the open position.

5. The tool of claim 1, wherein the plurality of first head fingers and the plurality of second head fingers are inter-digitated in the closed position.

6. The tool of claim 1, wherein the distraction head has a convex surface adapted to mate with an inferior facet and a concave surface adapted to mate with an superior facet.

7. The tool of claim 1, wherein the handle is pivotably actuatable about a pin, the pin being substantially perpendicular to a plane, wherein the first and second head components are configured to move along the plane when the handle is actuated.

8. The tool of claim 1, wherein the distraction head is shaped to contour a superior facet and an inferior facet of the facet joint.

9. A distraction tool to distract adjacent facets in a spine for insertion of an implant comprising:
a. a distraction head having a first head component and a second head component;
b. Two or more fingers extending from the first head component and one or more fingers extending from the second head component; wherein the two or more fingers of the first head component inter align with the one or more fingers of the second head component allowing the first head component and the second head component to be coplanar in the closed position; and
c. an actuatable handle coupled to the distraction head, wherein the plurality of fingers of the first head component and the plurality of fingers of the second head component remain parallel when the handle is operated to actuate the first and second head components to an open position.

10. The tool of claim 9, wherein the handle includes a first arm coupled to the first head component and a second arm coupled to the second head component, wherein the first arm longitudinally moves in relation to the second arm when the handle is actuated.

11. The tool of claim 9, wherein the plurality of first head fingers and the plurality of second head fingers are inter-digitated in the closed position

12. The tool of claim 9, wherein the distraction head has a convex surface adapted to mate with an inferior facet and a concave surface adapted to mate with an superior facet.

13. The tool of claim 10, wherein the distraction head is shaped to contour a superior facet and an inferior facet of the facet joint.

14. A distraction tool to distract adjoining facets of a spine for an implant comprising:
a. a distraction head including a first head component and a second head component, the first head component including a first set of fingers and the second head component including a second set of fingers, wherein the first set and the second set of fingers are inter-digitated when the distraction head is in a non-distracted position;
b. a handle attached to the distraction head, the handle actuatable to move the distraction head to a distracted position, wherein the first set of fingers and the second set of fingers are separated in the distracted position.

15. The tool of claim 14, wherein the second arm includes a longitudinal slot to accept a wedged portion of the first arm.

16. The tool of claim 14, wherein the first and second head components are adapted to contour a superior facet and an inferior facet of the spine.

17. The tool of claim 14, wherein the distraction head has a first surface adapted to mate with a superior facet and a second surface adapted to mate with an inferior facet, wherein the first and second surfaces have an arcuate shape.

18. The tool of claim 14, wherein the first handle is actuatable about a pin substantially perpendicular to a plane, wherein the first and second heads are configured to move along the plane when the first handle is actuated about the pin.

19. The tool of claim 14, wherein the distraction head is shaped to contour a superior facet and an inferior facet of the facet joint.

20. The tool of claim 14, wherein the first set of fingers and the second set of fingers remain approximately parallel to each other in the open position.
